(19) United States
(12) Patent Application Publication
(10) Pub. No.: US 2023/0253103 A1
(43) Pub. Date: Aug. 10, 2023

(54) SYSTEMS AND METHODS FOR MONITORING USER ACTIVITY

(71) Applicant: ResMed Sensor Technologies Limited, Dublin (IE)

(72) Inventors: Jose Ricardo Dos Santos, San Diego, CA (US); Redmond Shouldice, Dublin (IE)

(21) Appl. No.: 17/999,981

(22) PCT Filed: May 28, 2021
(86) PCT No.: PCT/US2021/034758

§ 371 Date: Nov. 28, 2022

Related U.S. Application Data
(60) Provisional application No. 63/031,485, filed on May 28, 2020.

(51) Int. Cl. G16H 40/63; G08B 21/18; G06F 3/01; A61B 5/11; A61B 5/0205; A61B 5/05

(52) U.S. Cl. CPC G16H 40/63; G08B 21/18; G06F 3/015; A61B 5/1118; A61B 5/0205; A61B 5/05; A61B 5/024

(57) ABSTRACT

A method includes causing first media content to be displayed on a display device, the first media content including a first prompt to perform a first activity. The method also includes receiving data associated with the user, the data including motion data associated with movement of the user and physiological data associated with the user. The method also includes determining whether the user is performing the first activity based at least in part on the physiological data, the motion data, or both. The method also includes determining a first physiological parameter associated with the user subsequent to the first prompt based at least in part on the physiological data associated with the user. The method also includes determining whether the first physiological parameter exceeds a predetermined threshold.

### CROSS-REFERENCE TO RELATED APPLICATIONS

This application claims the benefit of, and priority to, U.S. Provisional Patent Application No. 63/031,485 filed on May 28, 2020, which is hereby incorporated by reference herein in its entirety.

### TECHNICAL FIELD

The present disclosure relates generally to systems and methods for monitoring user activity, and more particularly, to systems and methods for determining whether a user is performing an activity.

### BACKGROUND

Patients are often prescribed virtual remote activity sessions (e.g., physical therapy, rehabilitation, exercises, breathing exercises, dance, meditation, etc.). Because these activity sessions are delivered virtually or remotely, the prescribing provider cannot directly observe and verify whether the patient is complying with the prompted activities. Similarly, the provider cannot directly verify whether the patient's vitals (e.g., heart rate, respiration rate, etc.) are within a safe range when the patient is performing the various activities. The present disclosure is directed to solving these and other problems.

### SUMMARY

According to some implementations of the present disclosure, a method includes causing first media content to be displayed on a display device, the first media content including a first prompt to perform a first activity. The method also includes receiving, from one or more sensors, data associated with the user, the data including (i) motion data associated with movement of the user and (ii) physiological data associated with the user. The method also includes determining whether the user is performing the first activity based at least in part on the motion data, the physiological data, or both. The method also includes determining a first physiological parameter associated with the user subsequent to the first prompt based at least in part on the physiological data associated with the user. The method also includes determining whether the first physiological parameter exceeds a predetermined threshold.

According to some implementations of the present disclosure, a system includes a one or more sensors configured to generate data associated with a user, a memory storing machine-readable instructions and media content, and a control system. The control system includes one or more processors configured to execute the machine-readable instructions to cause a display device to display a first portion of the media content, the first portion of the media content including a first prompt for performing a first activity. The control system is further configured to determine whether the user is performing the first activity based at least in part on the data generated by the one or more sensors. The control system is further configured to determine a first physiological parameter associated with the user based at least in part on the data generated by the one or more sensors. The control system is further configured to determine whether the first physiological parameter exceeds a predetermined threshold.

According to some implementations of the present disclosure, a device includes an interface configured to be received in a port of a display device, a memory storing machine-readable instructions and media content, and a control system. The control system is arranged to provide control signals to the display device via the interface. The control system includes one or more processors configured to execute the machine-readable instructions to cause the display device to display a first portion of the media content, the first portion of the media content including a first prompt for performing a first activity. The control system is further configured to receive data associated with a user from a sensor. The control system is further configured to determine whether the user is performing the first activity based at least in part on the data generated by the sensor. The control system is further configured to determine a first physiological parameter associated with the user based at least in part on the data generated by the sensor. The control system is further configured to cause the display device to display an indication of the determined first physiological parameter.

According to some implementations of the present disclosure, a method includes prompting a user to initiate an activity session comprising a first activity module and a second activity module. The first activity module and the second activity module are selected from a plurality of activity modules. The second activity module being subsequent to the first activity module. The method also includes receiving physiological data associated with the user during the first activity module. The method also includes receiving motion data associated with movement of the user during the first activity module. The method also includes modifying the activity session based at least in part on at least a portion of the physiological data associated with the user, at least a portion of the motion data associated with movement of the user, or both, wherein the modifying the activity session includes (i) pausing the first activity module for a first duration, (ii) pausing the activity session between the first module and the second module for a second duration, (iii) substituting the second activity module with a third activity module selected from the plurality of modules, or (iv) any combination thereof.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2A is a plan view of a dongle of the system of FIG. 1, according to some implementations of the present disclosure;

FIG. 2B is a plan view of a remote control of the system of FIG. 1, according to some implementations of the present disclosure;

FIG. 3A is a perspective view of at least a portion of the system of FIG. 1 and a user, according to some implementations of the present disclosure;

FIG. 3B is a side view of at least a portion of the system of FIG. 1, according to some implementations of the present disclosure;

FIG. 4 is a flow diagram for a method for determining whether a user is performing an activity, according to some implementations of the present disclosure;

FIG. 5 is a process flow diagram for a method according to some implementations of the present disclosure;

FIG. 6A is a schematic illustration of an activity session including a first activity module, a second activity module, and a third activity module, according to some implementations of the present disclosure;

FIG. 6B is a schematic illustration of a first modified activity session including the first activity module, a fourth activity module, and a fifth activity module, according to some implementations of the present disclosure;

FIG. 6C is a schematic illustration of a second modified activity session including a first pause in the first activity module and a second pause between the first activity module and the second activity module, according to some implementations of the present disclosure; and

FIG. 6D is a schematic illustration of a third modified activity session including the first activity session and a fourth activity session, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Individuals are often prescribed virtual remote activity sessions (e.g., physical therapy, exercises, rehabilitation, pulmonary rehabilitation, etc.) that can be performed at home or another location outside of a traditional medical or therapy facility without direct observation by the provider (e.g., doctor, nurse, therapist, etc.) or another professional. The virtual activity session can include a recorded video of an individual performing certain movements or exercises that can be displayed on a display device (e.g., television, computer, tablet, smart phone, etc.). The user is generally prompted to observe the activities in the video and follow along with the movements to perform the activity.

Such virtual activity sessions are often delivered to seniors who may not be technologically proficient in using internet-connected devices (e.g., smartphones, tablets, laptops, etc.) or even own such devices. For example, the user may be a senior citizen living in a facility such as a nursing home, assisted living, retirement community, etc. These facilities often have televisions for each resident. Thus, it would be advantageous to deliver the virtual therapy content through a device that can plug into the television and automatically display the required content with little or no action required by the user to initiate the media content.

Because the activity session is delivered remotely (e.g., as opposed to in-person), the provider cannot directly observe and verify whether the user is complying with the prompted activities. For example, the activity may be too difficult for the user or the user may not be sufficiently motivated to perform the activity correctly (or at all) if there is no mechanism to verify compliance. While a camera could be used to record the user during the session to verify that the user complied with the activity, a third party would need to watch the video or review images to determine whether and how the user performed the activity. Further, using a camera to record the user performing the activity is intrusive and could raise privacy concerns. For example, the user may find it undesirable to have another person potentially watching a video of them performing physical activity and/or seeing the inside of their home or living space, which could reveal personal information.

Similarly, in an in-person setting, the provider can directly observe or verify the user's vitals such as heart rate, heart rate variability, cardiac waveform, respiration rate, respiration rate variability, respiration depth, perspiration, temperature (e.g., ambient temperature, body temperature, core body temperature, surface temperature, etc.), blood oxygenation, photoplethysmography, pulse transmit time, blood pressure or any combination thereof to determine whether the user is over-exerting themselves and/or performing the activity safely. This monitoring is especially important for seniors, where overexertion can pose a serious health risk (e.g., heart attack, stroke, injury, falling, etc.). Thus, it would be advantageous to automatically monitor physiological data associated with the user during the activity to determine whether there is a safety risk. It would be further advantageous to automatically modify (e.g., pause or stop) the displayed content in response to determining that the physiological parameters (e.g., heart rate, breathing rate, etc.) exceed a predetermined threshold. The present disclosure is directed to solving these and other problems.

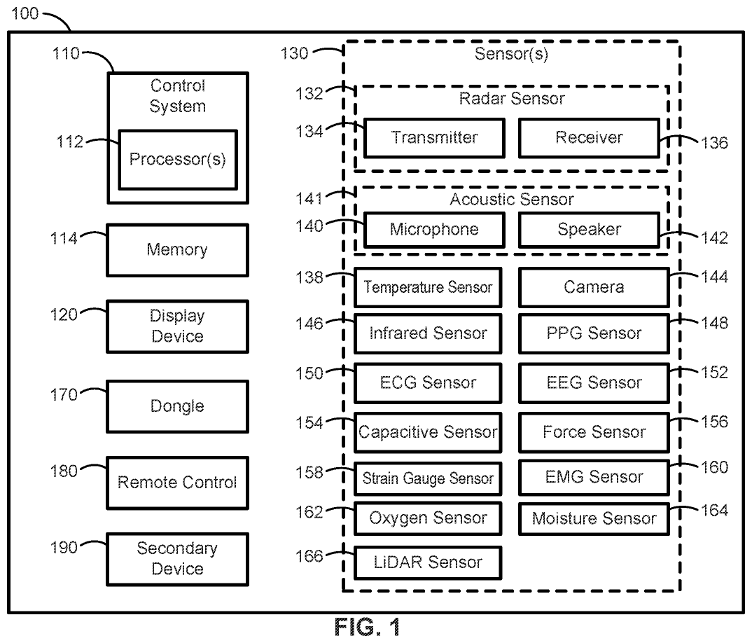

Referring to FIG. 1, a system 100, according to some implementations of the present disclosure, is illustrated. The system 100 includes a control system 110, a memory device 114, a display device 120, and one or more sensors 130. In some implementation, the system 100 optionally includes a dongle 170, a remote control 180, and/or a secondary device 190.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the display device 120, within a housing of the dongle 170, within a housing of the remote control 180, within a housing of the secondary device 190, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the display device 120, within a housing of the dongle 170, within a housing of the remote control 180, within a housing of the secondary device 190, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia, an employment status of the user, an educational status of the user, a socioeconomic status of the user, or any combination thereof. The medical information can include, for example, including indicative of one or more medical conditions associated with the user, medication usage by the user, or both. The medical information can also include a fall risk assessment associated with the user (e.g., a fall risk score using the Morse fall scale). The medical information data can further include a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof. In some implementations, the memory device 114 stores media content that can be displayed on the display device 120.

While the control system 110 and the memory device 114 are described and shown in FIG. 1 as being a separate and distinct component of the system 100, in some implementations, the control system 110 and/or the memory device 114 are integrated in the display device 120, the dongle 170, the remote control 180, the secondary device 190, or any combination thereof. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

As described herein, the processor 112 and/or memory device 114 can receive data (e.g., physiological data and/or motion data) from the one or more sensors 130 such that the data for storage in the memory device 114 and/or for analysis by the processor 112. The processor 112 and/or memory device 114 can communicate with the one or more sensors 130 using a wired connection or a wireless connection (e.g., using an RF communication protocol, a Wi-Fi communication protocol, a Bluetooth communication protocol, over a cellular network, etc.). In some implementations, the system 100 can include an antenna, a receiver (e.g., an RF receiver), a transmitter (e.g., an RF transmitter), a transceiver, or any combination thereof. Such components can be coupled to or integrated a housing of the control system 110 (e.g., in the same housing as the processor 112 and/or memory device), the display device 120, the dongle 170, the remote control 180, or the secondary device 190.

The display device 120 is generally used to display image(s) including still images, video images, or both. The display device 120 can be, for example, a television (e.g., a smart television), a smart phone, a tablet, a laptop, a monitor, or the like. In some implementations, the display device 120 acts as a human-machine interface (HMI) that includes a graphic user interface (GUI) configured to display the image(s) and an input interface. The display device 120 can be an LED display, an OLED display, an LCD display, or the like. The input interface can be, for example, a touchscreen or touch-sensitive substrate, a mouse, a keyboard, or any sensor system configured to sense inputs made by a human user interacting with the display device 120. In some implementations, one or more display devices can be used by and/or included in the system 100.

The one or more sensors 130 of the system 100 include a radar sensor 132, temperature sensor 138, a microphone 140, a speaker 142, a camera 144, an infrared sensor 146, a photoplethysmogram (PPG) sensor 148, an electrocardiogram (ECG) sensor 150, an electroencephalography (EEG) sensor 152, a capacitive sensor 154, a force sensor 156, a strain gauge sensor 158, an electromyography (EMG) sensor 160, an oxygen sensor 162, a moisture sensor 164, a LiDAR sensor 166, a ballistocardiogram sensor, or any combination thereof. Generally, each of the one or sensors 130 are configured to output sensor data (e.g., including motion data and/or physiological data associated with a user) that is received and stored in the memory device 114 or one or more other memory devices.

While the one or more sensors 130 are shown and described as including each of the radar sensor 132, the temperature sensor 138, the microphone 140, the speaker 142, the camera 144, the infrared sensor 146, the photoplethysmogram (PPG) sensor 148, the electrocardiogram (ECG) sensor 150, the electroencephalography (EEG) sensor 152, the capacitive sensor 154, the force sensor 156, the strain gauge sensor 158, the electromyography (EMG) sensor 160, the oxygen sensor 162, the moisture sensor 164, and the LiDAR sensor 166 more generally, the one or more sensors 130 can include any combination and any number of each of the sensors described and/or shown herein.

The radar sensor 132 includes a transmitter 134 and a receiver 136 and is generally used to generate motion data associated with a user, physiological data associated with a user, or both. The transmitter 134 generates and/or emits radio waves having a predetermined frequency and/or a predetermined amplitude (e.g., within a high frequency band, within a low frequency band, long wave signals, short wave signals, etc.). The receiver 136 detects the reflections of the radio waves emitted from the transmitter 134, and this data can be analyzed by the control system 110 to determine movement and/or physiological parameters associated with a user, for example. The specific format of the RF communication can be Wi-Fi, Bluetooth, or the like. An RF receiver (either the RF receiver and the RF transmitter or another RF pair) can also be used for wireless communication between the control system 110, the one or more sensors 130, the display device 120, or any combination thereof In some such implementations, the radar sensor 132 also includes a control circuit.

In some implementations, the radar sensor 132 is an ultra-wide-band (UWB) radar sensor. UWB uses a low energy level for short-range, high-bandwidth communication (e.g., at a frequency greater than 500 MHz). The signals emitted by the UWB radar sensor can pass through various obstacles (e.g., the display device 120) that are positioned between and/or block a direct line of sight between, for example, a user and the radar sensor 132. The radar sensor 132 can be an FMCW (Frequency Modulated Continuous Wave) based system or system on chip where the frequency increases linearly with time (e.g., a chirp) with different shapes such as triangle (e.g., frequency swept up, then down), sawtooth (e.g., frequency ramp swept up or down, then reset), stepped or non-linear shape and so forth. The radar sensor 132 can use multiple chirps that do not overlap in time or frequency, with one or more transmitters and receivers, and can operate at or around any suitable frequencies, such as at or around 24 GHz, or at or around millimeter wave (e.g., between about 76-81 GHz) or similar frequencies. The radar sensor 132 can measure range as well as angle and velocity.

In some implementations, data from the radar sensor 132 can be used to generate motion data that is indicative of, for example, movement(s) of the user (e.g., during an activity), including gait, falls, behavior, etc. Using the radar sensor 132 to detect movement of a user is advantageous compared to, for example, the camera 144 because the radar sensor 132 does not obtain images of the user and/or other personal information. In some implementations, data from the radar sensor 132 can also be used to determine one or more physiological parameters associated with the user, such as, for example, heart rate, heart rate variability, cardiac waveform, respiration rate, respiration rate variability, respiration depth, perspiration, temperature (e.g., ambient temperature, body temperature, core body temperature, surface temperature, etc.), blood oxygenation, photoplethysmography, pulse transmit time, blood pressure or any combination thereof. In other implementations, data from the radar sensor 132 can be used to identify the user or verify the identity of the user (e.g., based on previously recorded data associated with the user).

In some implementations, the radar sensor 132 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include a receiver and/or transmitter that the is the same as, or similar to, the transmitter 134 and/or receiver 136. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. In some implementations, the motion sensor is a high frequency 5G mobile phone and/or base station that includes controller software therein to sense motion. For example, a node in a 5G network can be used for motion sensing, using subtle changes in RSS (receive signal strength) across multiple channels. Further, such motion sensors can be used to process motion from multiple targets, breathing, heart, gait, fall, behavior analysis, etc. across an entire home and/or building and/or hospital setting.

The temperature sensor 138 outputs temperature data that can be stored in the memory device 114 and/or analyzed by the processor 112 of the control system 110. In some implementations, the temperature sensor 138 generates temperatures data indicative of a core body temperature of the user, a skin temperature of the user, an ambient temperature, or any combination thereof. The temperature sensor 138 can be, for example, a thermocouple sensor, a thermistor sensor, a silicon band gap temperature sensor or semiconductor-based sensor, a resistance temperature detector, or any combination thereof.

The microphone 140 outputs sound data that can be stored in the memory device 114 and/or analyzed by the processor 112 of the control system 110. The microphone 140 can be used to record sound(s) during a sleep session (e.g., sounds from the user) to determine (e.g., using the control system 110) one or more sleep-related parameters, as described in further detail herein. The microphone 140 can be coupled to or integrated in the display device 120, for example. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones

The speaker 142 outputs sound waves that are audible to a user of the system 100 (e.g., the user). The speaker 142 can be used, for example, to communicate an alert or message to the user (e.g., in response to a physiological parameter of the user exceeding a predetermined threshold). The speaker 142 can be coupled to or integrated in the display device 120, for example.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user or their bed partner. Based at least in part on the data from the microphone 140 and/or the speaker 142, the control system 110 can determine, for example a location of the user and/or movement of the user. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety.

The camera 144 outputs image data reproducible as one or more images (e.g., still images, video images, thermal images, or a combination thereof) that can be stored in the memory device 114. The image data from the camera 144 can be used by the control system 110 to determine movement of the user (e.g., to determine whether the user is complying with therapy). For example, the image data from the camera 144 can be used in combination with data from the radar sensor 132 to determine whether the user is performing an activity (e.g., therapy). In some implementations, the system 100 excludes the camera 144 and generates motion data and/or physiological data only using the radar sensor 132 described above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The camera 144 can be a CCD (charge coupled device) sensor or a CMOS (complementary metal oxide semiconductor) sensor. The camera 144 can sense visible light (bright light or low light), near infrared, or infrared, thermal and so forth. The camera 144 can be an RGB (red green blue) camera, a multispectral near infrared (NIR) or hyperspectral camera, and can have a range of lenses and filters, with a range or focal lengths, or be varifocal. Camera processing can track motion from frame to frame, and perform face detection and automatic zooming to only process the face, or process chest movement after identifying (the presence of a or actual identification of) a person. Camera detection can include gross motion detection, motion classification, and detection of fine motions such as breathing and chest movement related to heart beat. Camera analysis can estimate temperature, sweatiness or perspiration (e.g., during a workout), and blood pressure by estimated pulse transit time (e.g., via video-based PPG).

The infrared (IR) sensor 146 outputs infrared image data reproducible as one or more infrared images (e.g., still images, video images, or both) that can be stored in the memory device 114. The infrared data from the IR sensor 146 can be used to determine, for example, a temperature of the user and/or movement of the user. The IR sensor 146 can also be used in conjunction with the radar sensor 132 and/or camera 144 when measuring the presence, location, and/or movement of the user. The IR sensor 146 can detect infrared light having a wavelength between about 700 nm and about 1 mm, for example, while the camera 144 can detect visible light having a wavelength between about 380 nm and about 740 nm. In some examples, the IR sensor 146 is a passive infrared (PIR) sensor.

The PPG sensor 148 outputs physiological data associated with the user that can be used to determine one or more physiological parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 148 can be worn by the user, embedded in clothing and/or fabric that is worn by the user or embedded in and/or coupled to the secondary device 190.

The ECG sensor 150 outputs physiological data associated with electrical activity of the heart of the user. In some implementations, the ECG sensor 150 includes one or more electrodes that are positioned on or around a portion of the user (e.g., during a therapy session). The physiological data from the ECG sensor 150 can be used, for example, to determine one or more of the physiological parameters described herein.

The EEG sensor 152 outputs physiological data associated with electrical activity of the brain of the user. In some implementations, the EEG sensor 152 includes one or more electrodes that are positioned on or around the scalp of the user. The physiological data from the EEG sensor 152 can be used, for example, to determine a sleep state of the user.

The capacitive sensor 154, the force sensor 156, and the strain gauge sensor 158 output data that can be stored in the memory device 114 and used by the control system 110 to determine one or more of the parameters described herein. The EMG sensor 160 outputs physiological data associated with electrical activity produced by one or more muscles. The oxygen sensor 162 outputs oxygen data indicative of an oxygen concentration of gas. The oxygen sensor 162 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The moisture sensor 164 outputs data that can be stored in the memory device 114 and used by the control system 110. The moisture sensor 164 can be used to detect moisture in various areas surrounding the user. In some implementations, the moisture sensor 164 can be coupled to or integrated in the secondary device 190 or the clothing of the user to monitor perspiration of the user. The moisture sensor 164 can also be used to monitor the humidity of the ambient environment surrounding the user, for example, the air inside the room where the user is performing a virtual therapy session.

The Light Detection and Ranging (LiDAR) sensor 166 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 166 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the display device 120, the dongle 170, the remote control 180, the secondary device 190, or any combination thereof. For example, the radar sensor 132 can be integrated in and/or coupled to the dongle 170 or the remote control 180. In some implementations, at least one of the one or more sensors 130 is not coupled to the display device 120 and is positioned generally adjacent to the user (e.g., positioned on or in contact with a portion of the user, worn by the user, coupled to or positioned on furniture, coupled to the ceiling or a wall, etc.).

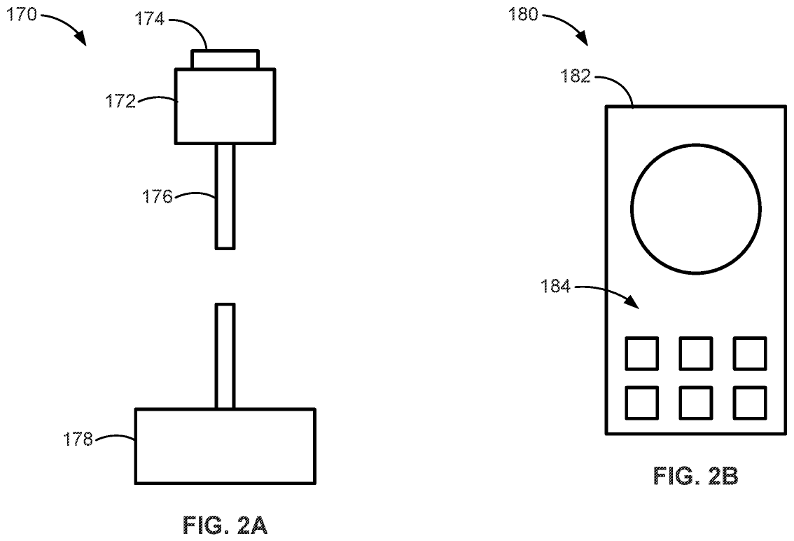

In implementations of the system 100 including the dongle 170, the dongle 170 is generally used to store media content (e.g., virtual therapy sessions) that can be displayed via, for example, the display device 120. Referring to FIG. 2A, the dongle 170 includes a first housing portion 172, an interface 174, a cord 176, and a second housing portion 176. The interface 174 is at least partially received within a portion (e.g., port) of the display device 120 to communicatively couple the dongle 170 to the display device 120. The interface 174 can be received in, for example, an HDMI port, a mini HDMI port, a USB port, a micro-USB port, a mini-USB port, a USB-A port, a USB-B port, a USB-C port, a DisplayPort, a mini DisplayPort, a VGA port, a mini-VGA port, an S-video port, composite port, a component port, etc. Thus, the dongle 170 can cause media content stored thereon to be displayed on the display device 120.

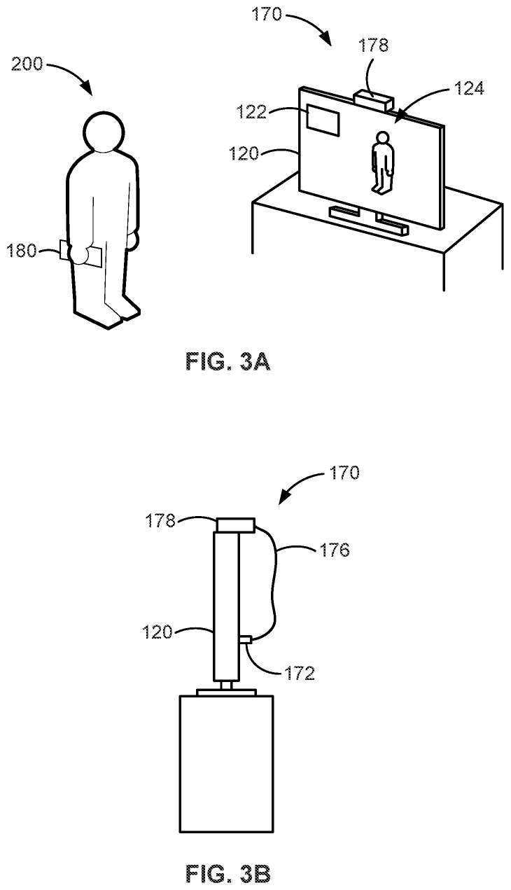

The cord 176 communicatively couples the interface 174 and/or other components in the first housing portion 172 with components in the second housing portion 178. The second housing portion 178 includes a clip for coupling the dongle 170 to a portion of the display device 120. For example, referring to FIGS. 3A and 3B, the second housing portion 178 of the dongle 170 can be coupled to a first portion of the display device 120 (e.g., a bezel, a frame, an outer edge, a stand, etc.), while the interface 174 is coupled to a second portion (e.g., a rear or side input port) of the display device 120 or another device that provides video input to the display device 120 (e.g., a receiver or cable box). While the dongle 170 is shown as being coupled to an upper edge of the display device 120 in FIG. 3A, more generally the dongle 170 can be coupled to any portion of the display device 120 that does not substantially obstruct the user 200 from viewing media content on the display device 120 (e.g., coupled to either side or the lower edge of the display device 120). In another alternative, the dongle 170 is not coupled to the display device 120 and is positioned generally adjacent to the display device 120 (e.g., on a media console, a shelf, or other furniture).

In some implementations, the components of the dongle 170 are powered by the display device 120 via the interface 174. In other implementations, the dongle 170 includes a second cord that delivers power to the components therein from, for example, an AC power outlet or a USB port.

As described above, at least one of the sensor(s) 130, the control system, and/or the memory device 114 are coupled to or integrated in the dongle 170. The memory device 114 stores media content that can be displayed on the display device 120 and machine readable instructions that are executable by the control system 110 to perform any of the functions described herein. More specifically, the sensor(s) 130 can be coupled to or integrated in the second housing portion 178 so that the sensor(s) 130 have a direct line of sight to the user 200 that is positioned generally in front of the display device 120. For example, the radar sensor 132 (FIG. 1) described above can be integrated in the second housing portion 178 of the dongle 170 such that the radar sensor 132 can generate motion data associated with movement of the user 200, physiological data associated with the user 200, or both.

In some implementations, the dongle 170 can communicate (e.g., over the Internet via Wi-Fi) with one or more remote devices or servers. For example, the dongle 170 can communicate with a remote server to receive new or updated media content to store in the memory device 114. As another example, the dongle 170 can communicate with the remote server to transmit the data associated with the user performing described herein (e.g., to a medical provider associated with the user). As a further example, the dongle 170 can communicate with the remote server to receive a software update or patch. As yet a further example, the dongle 170 can communicate with a device associated with the user that is separate and distinct from the display device 120 when the display device 120 is a television (e.g., a smartphone, a tablet, a laptop, etc.).

In some implementations, the dongle 170 does not include the cord 176 and the second housing portion 178. In such implementations, the dongle 170 can be referred to as a “media stick” and the memory 114 and/or the sensor(s) 130 are positioned in the first housing portion 172.

While the dongle 170 is described and shown herein as being communicatively coupled to the display device 120 via the interface 174 via a direct wired connection, in some implementations, the dongle 170 is communicatively coupled to the display device 120 via a wireless connection (e.g., using Wi-Fi or Bluetooth).

The remote control 180 is generally used to control and/or actuate the display device 120, the dongle 170, or both. Referring to FIG. 2B, the remote control 180 includes a housing 182 and a plurality of user-selectable buttons 184 for controlling the functions of the display device 120 and/or the dongle 170. That is, the remote control 180 is wirelessly communicatively coupled to the display device 120 and/or the dongle 170.

The plurality of user-selectable buttons 184 can control the displayed media content by causing the display device 120 and/or dongle 170 to perform, for example, a power on operation, a power off operation, a volume up operation, a volume down operation, a mute operation, a play operation, a pause operation, a stop operation, a fast forward operation, a rewind operation, a next operation, a back operation, a record operation, a menu operation, or any combination thereof. The plurality of user-selectable buttons 184 can also include one or more buttons (e.g., a track pad, a touch pad, arrow keys, etc.) to permit the user 200 to select one or more user-selectable elements that are displayed on the display device 120. The plurality of user-selectable buttons 184 can also include a panic button that transmits an alert to a third party indicating the user 200 needs assistance.

In some implementations, at least one of the one or more sensors 130 (FIG. 1) are coupled to or integrated in the housing 182 of the remote control 180. For example, the radar sensor 132 can be coupled to or integrated in the housing 182 instead of the dongle 170. In this example, the remote control 180 can generally be positioned generally adjacent to the display device 120 (e.g., positioned on furniture adjacent to the display device 120). and collect data associated with a user that is positioned in front of the display device 120. As another example, the microphone 140 can be coupled to or integrated in the remote control 180 (e.g., to receive voice commands from the user). As a further example, the remote control 180 can include the infrared sensor 146 for checking a temperature of the user and/or the oxygen sensor 162 for checking an oxygen level of the user. As yet another example, the remote control 180 can include one or more accelerometers and/or gyroscopes that can be used to determine motion of the remote control 180 and/or movement of a user that is holding the remote control 180. The housing 182 can also include a power source for powering the remote control 180 (e.g., a rechargeable battery or a replaceable battery).

The secondary device 190 is separate and distinct from the display device 120, the dongle 170, and the remote control 180 and can include at least one of the one or more sensors 130 so that the secondary device 190 can generate motion data and/or physiological data associated with a user. The secondary device 190 can be positioned in the same room as the dongle 170 and display device 120, or a different room. For example, the display device 120 and dongle 170 can be in a first room (e.g., bedroom or living room), while the secondary device 190 can be positioned in a second room (e.g., a bathroom). In some implementations, the secondary device 190 includes the radar sensor 132 and can be plugged into a AC power outlet (e.g., in a bathroom). In other implementations, the secondary device 190 is a wearable device (e.g., a smart watch or bracelet) that can be worn or donned by a user. In such implementations, one or more of the sensors 130 described herein can be integrated or embedded in the wearable secondary device 190.

In implementations of the system 100 that do not include the dongle 170 or the remote control 180, at least one of the one or more sensors 130 can be coupled to or integrated in the display device 120 and can perform the same functions as the sensor(s) described herein as being coupled to or integrated in the dongle 170. For example, in such implementations, the radar sensor 132 can be coupled to or integrated in the display device 120 (e.g., in or on a bezel) for capturing motion data and/or physiological data associated with a user.

While system 100 is shown as including all of the components described above, more or fewer components can be included in a system for generating monitoring compliance with an activity and/or physiological parameters associated with the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the display device 120. As yet another example, a third alternative system includes the control system 110, the memory device 114, the display device 120, at least one of the one or more sensors 130, the dongle 170, and the remote control 180. Thus, various systems can be formed using any portion or portions of the components shown and described herein and/or in combination with one or more other components.

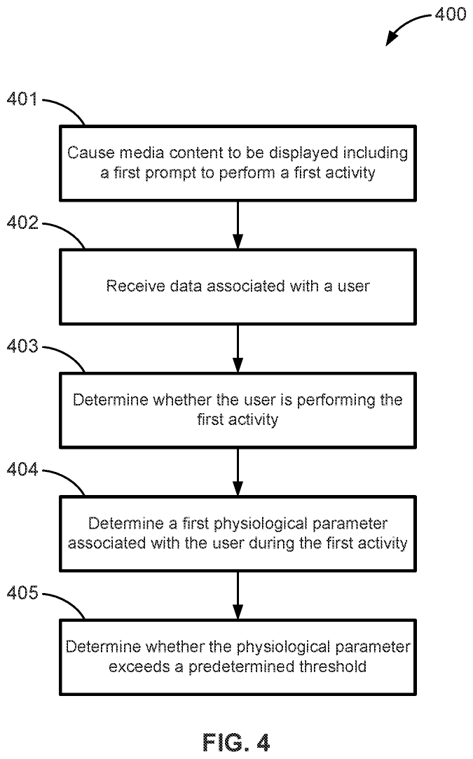

Referring to FIG. 4, a method 400 for determining user compliance with an activity according to some implementations of the present disclosure is illustrated. One or more steps of the method 400 can be implemented using any element or aspect of the system 100 (FIGS. 1-3B) described herein.

Step 401 of the method 400 includes causing first media content to be displayed on the display device 120. The first media content can include one or more videos, one or more images, audio, or any combination thereof. In some implementations, the first media content includes one or more prompts for a user to perform a first activity, such as, for example, physical therapy, rehabilitation, exercises, breathing exercises, dance, meditation, etc. The first media content can be stored on, for example, the memory device 114 (FIG. 1), which can be coupled to or integrated in the dongle 170. In one example, the control system 110 (FIG. 1) executes machine-readable instructions stored in the memory device 114 to cause the dongle 170 to play the first media content on the display device 120 responsive to the interface 174 being coupled to the display device 120. As another example, an input (e.g., manual input from the user) on the remote control 180 can cause the dongle 170 to play the first media content on the display device 120. In another example, the control system 110 (FIG. 1) executes machine-readable instructions stored in the memory device 114 to cause the dongle 170 to automatically play the first media content on the display device 120 at a predetermined time (e.g., every day at 1 PM). In some implementations, step 401 includes authenticating and/or authorizing a user prior to causing the dongle 170 to play media content on the display device 120 (e.g., using a username/password, a PIN, biometrics, etc.).

In some implementations, the first prompt is a recorded video of an individual performing the first activity that the user can follow along with. For example, an individual 124 is displayed on the display device 120 in FIG. 3A. The individual 124 can be someone other than the user (e.g., an instructor) performing the first activity or a previously recorded video the user performing the first activity. In some implementations, the individual 124 is an avatar (e.g., a representation of a person) that performs the first activity so that the user follows along. In such implementations, an image of at least a portion of the user (e.g., the face of the user) can be overlaid on a portion of the avatar. In other implementations, the first prompt additionally or alternatively includes alphanumeric text that is displayed on the display device 120 and/or audio that is communicated to the user (e.g., via the speaker) to prompt the user to perform the first activity.

In some implementations, the displayed first media content can include a chat interface to permit the user to communicate with third parties (e.g., facility or home health staff nurse, family, a centralized training monitor, etc.). In other implementations, the displayed first media content can include one or more images and/or videos associated with the user (e.g., family photos or videos). In such implementations, these images and/or videos can relax or distract the user and aid in reduce symptoms of depression and/or agitation (e.g., reminiscence therapy).

While the first media content is generally described herein as being displayed via a television, smartphone, tablet, laptop, etc., in some implementations, the first media content can be delivered or displayed using a virtual or augmented reality system (e.g., a virtual reality headset).

Step 402 of the method 400 includes generating and/or receiving first data associated with user while the first media content is being display on the display device 120. The first data includes motion data indicative of movement of the user (or the lack thereof) and/or physiological data indicative of one or more physiological parameters of the user such as, for example, heart rate, heart rate variability, cardiac waveform, respiration rate, respiration rate variability, respiration depth, a tidal volume, an inspiration amplitude, an expiration amplitude, an inspiration-expiration ratio, perspiration, temperature (e.g., ambient temperature, body temperature, core body temperature, surface temperature, etc.), blood oxygenation, photoplethysmography, pulse transmit time, blood pressure, or any combination thereof. The first data can be generated by any one of the sensor(s) 130 described herein. In some implementations, the motion data and the physiological data are both generated by the radar sensor 132 (FIG. 1), where the radar sensor 132 is coupled to or embedded in dongle 170 or the remote control 180. In other implementations, the motion data is generated and/or received from a first one of the sensors 130 and the physiological data is generated and/or received from a second one of the sensors 130 that is different than the first one of the sensors 130. The first data can be received by and stored in the memory device 114, for example.

In some implementations, step 402 includes verifying a presence of a user and/or an identity of the user. For example, step 402 can include determining a presence of a user based on whether the user is within a predetermined field of view of at least one of the one or more sensors 130. The identity of the user can be verified by the control system 110 using data from at least one of the sensors 130, such as the radar sensor 130, the camera 144 (e.g., using a facial recognition algorithm), the microphone 140 (e.g., using a voice recognition algorithm), or any combination thereof In other implementations, the user can be prompted to provide authentication and/or authorization information (e.g., username, password, PIN, etc.) via the display device 120.

Step 403 of the method 400 includes determining whether the user is performing the first activity based at least in part on the first data (step 402). As described above, in some examples, the first media content includes a previously recorded video of an individual or avatar performing the first activity to prompt the user to perform the first activity. Thus, step 403 can include comparing the motion data associated with the user (step 402) with previously recorded motion data to determine whether the user is performing the first activity.

In some implementations, step 403 includes analyzing the data using a machine learning algorithm to determine whether the user is performing the first activity. In such implementations, the machine learning algorithm can be trained (e.g., using previously recorded motion data from a plurality of users) to receive current motion data as an input and output a determination whether the user is performing the first activity. In some implementations, step 403 can include determining a percentage match between the current motion data associated with the user and the previously recorded motion data. The user is performing the first activity if the percentage match exceeds a predetermined threshold (e.g., at least 60%, at least 75%, at least 90%, etc.). In some examples, step 403 includes determining a range of motion of the user based at least in part on the motion data (step 402).

In some implementations, the determination whether the user is performing the first activity in step 403 is a binary decision (e.g., yes or no). In other implementations, step 403 includes determining a degree to which the user is performing the first activity. For example, step 403 can include determining a percentage of the first activity that the user is performing (e.g., 10%, 50%, 90%, etc.). In some implementations, step 403 includes measuring a performance of the user while performing first activity (e.g., poor, fair, good, excellent, etc.).

In some implementations, responsive to determining that the user is not performing the first activity, the method 400 can include generating an alert and communicating the alert to the user or a third party. For example, the alert can be communicated to the user via the display device 120 and/or the speaker 142. As another example, a second prompt to perform the first activity can be communicated to the user responsive to determining that the user is not performing the first activity. The second prompt can be the same as, or different than, the first prompt. In yet another example, the method 400 can include modifying an operation of the display device 120 responsive to determining that the user is not performing the first activity (e.g., pausing the or stopping the media content displayed on the display device 120). In a further example, the method 400 can include modifying the media content to display a second prompt to perform a second activity that is different than the first activity responsive to determining that the user is not performing the first activity.

Step 404 of the method 400 includes determining a first physiological parameter associated with the user based at least in part on the first data (step 402). For example, the memory device 114 can include machine-readable instructions that when executed by the control system 110 cause the control system 110 to analyze the physiological data (step 402) to determine the first physiological parameter. The physiological parameter can be, for example, a heart rate, a heart rate variability, a cardiac cycle, respiration rate, an inspiration amplitude, an expiration amplitude, an inspiration-expiration ratio, estimated blood pressure parameter(s), or any combination thereof. More generally, step 404 can include determining any number of physiological parameters (e.g., a plurality of parameters). Information indicative of the determined first physiological parameter can be stored in the memory device 114.

In some implementations, step 404 includes displaying one or more indications of the determined first physiological parameter on the display device or otherwise communicating an indication of the determined first physiological parameter to the user. In this manner, the user can see the determined first physiological parameter in substantially real-time while performing the first activity. Further, this can aid in assuring the user that they are safely performing the first activity. For example, referring to FIG. 3A, a first indication 122 is displayed on the display device 120. The first indication 122 can include alphanumeric text, symbols, images, audio, or any combination thereof. In some examples, the first indication 122 is displayed adjacent to the first media content (e.g., adjacent to the individual 124). In other examples, the first indication 122 is at least partially overlaid on a portion of the displayed first media content.

Step 405 of the method 400 includes determining whether the determined first physiological parameter (step 404) exceeds a predetermined threshold. In some implementations, the predetermined threshold for the first physiological parameter is indicative of an unsafe or unhealthy level. For example, if the first physiological parameter is a heart rate, the threshold can be indicative of a high heart rate (e.g., over 120 BPM, over 130 BPM, over 150 BPM, over 175 BPM, over 200 BPM, over BPM 250, etc.) that could pose a health risk to the user. In some examples, the predetermined threshold is a range of values.

In some implementations, step 405 includes determining and/or adjusting the predetermined threshold based on information associated with the user. For example, the predetermined threshold can be determined and/or adjusted for the user based at least in part on the user profile described above (e.g., demographic information, medical information, fall risk information, etc.). If the predetermined threshold is a heart rate, for example, a safe threshold may depend on the age of the user and/or medical conditions. Safe ranges can be obtained using a look-up table stored in the memory device 114.

In some implementations, responsive to determining that the first determined physiological parameter exceeds the predetermined threshold during the first activity, the method 400 can include generating an alert and communicating the alert to the user or a third party. For example, the alert can be communicated to the user via the display device 120 and/or the speaker 142. As another example, the alert can be communicated to a family member or medical provider/staff to prompt them to check on the third party. As yet another example, the alert can be communicated to emergency services. In some implementations, the alert is a prompt or feedback (e.g., alphanumeric text or other visual cues displays on the display device 120 and/or audio) encouraging the user to modify their behavior and/or movements. For example, the alert can encourage the user reduce their exertion (e.g., slow down) to aid in causing modifying the physiological parameter (e.g., so that it no longer exceeds the predetermined threshold).

In other implementations, responsive to determining that the first determined physiological parameter exceeds the predetermined threshold during the first activity, the method 400 can include modifying an operation of the display device 120. For example, the control system 110 can pause or stop the displayed first media content to stop the user from continuing to perform the first activity. In such implementations, steps 402-404 can be repeated one or more times until it is determined that the physiological parameter no longer exceeds the predetermined threshold (e.g., the heart rate has returned to a safe level). In response to determining the physiological parameter no longer exceeds the predetermined threshold, the first media content can resume and the user can continue performing the first activity. Alternatively, the first media content will no longer be displayed after the physiological parameter returns to a safe level. Instead, the control system 110 can cause second media content to be displayed on the display device 120 including a second prompt to perform a second activity that is different than the first activity (e.g., having a lower difficulty than the first activity) to aid in preventing the physiological parameter from exceeding the predetermined threshold again.

In some implementations, the method 400 includes communicating one or more messages to the user (e.g., via the display device 12) while performing the first activity to encourage the user to perform the first activity. The messages can include alphanumeric text, audio, images, pre-recorded videos, or any combination thereof. In such implementations, the messages can be customized to include the name of the user.

In some implementations, the method 400 includes using a learning artificial intelligence (AI) system (e.g., using a deep neural network) to analyze the first media content (e.g., video stream) and metadata to classify the image data, then checks the compliance and engagement of the user to the media content, and the related physiological parameters (e.g., they are able to safely exercise in their optimized breathing and cardiac biometrics zone such as to have good exercise with low risk), and select a similar type of media (but with fresh content) for future sessions, such that the users remains engaged with the program.

In some implementations, one or more of steps 401-405 can be repeated one or more times. For example, steps 402-405 can be repeated one or more times until it is determined that the user has completed the first activity, at which point the first media content is no longer displayed on the display device. In some examples, steps 401-405 are repeated after the first activity is performed for a second activity, where second media content is displayed on the display device including a second prompt for performing a second activity. In some implementations, the second activity is different than the first activity. For example, the second activity can have a difficulty that is different than a difficulty of the first activity. For example, if it is determined that the user satisfactorily performed the first activity, the second activity can have a difficulty that is greater than a difficulty of the completed first activity. In this manner, the user can be prompted to perform multiple activities.

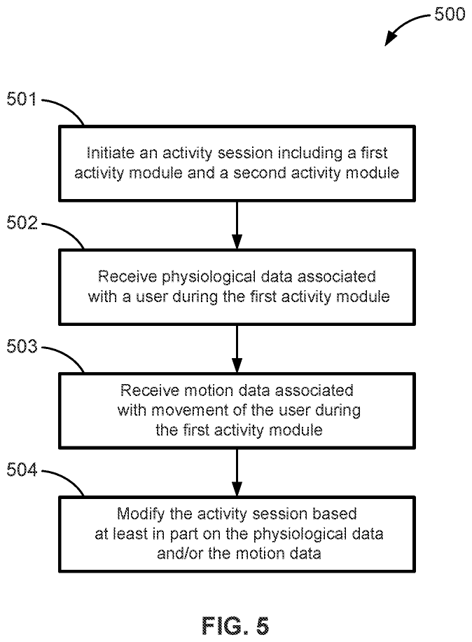

Referring to FIG. 5, a method 500 according to some implementations of the present disclosure is illustrated. One or more steps of the method 500 can be implemented using any element or aspect of the system 100 described herein.

Step 501 of the method 500 includes prompting a user to initiate an activity session. The activity session comprises at least one activity module that is selected from a plurality of activity modules. Generally, each activity module includes media content (e.g., video and/or audio) prompting the user to perform one or more physical activities. Such media content can be communicated to the user, for example, using the display device 120 (FIG. 1). The user can be prompted to initiate an activity module by causing an activity module to be displayed (e.g., via the display device 120). For example, an activity module can prompt the user to perform a series of activities such as muscle movements, movements of one or more parts of the body (e.g., arm movements, leg movements, head movements, etc.) a series of breathing exercises, or the like, or any combination thereof. Each activity module can include a pre-recorded video of another individual (e.g., coach or instructor) or avatar performing the activities, audio instructing the user to perform the activities, text instructing the user to perform the activities (e.g., subtitles), or any combination thereof. In this manner, the activity session can be considered, for example, a physical therapy session, a pulmonary therapy session, or an exercise session.

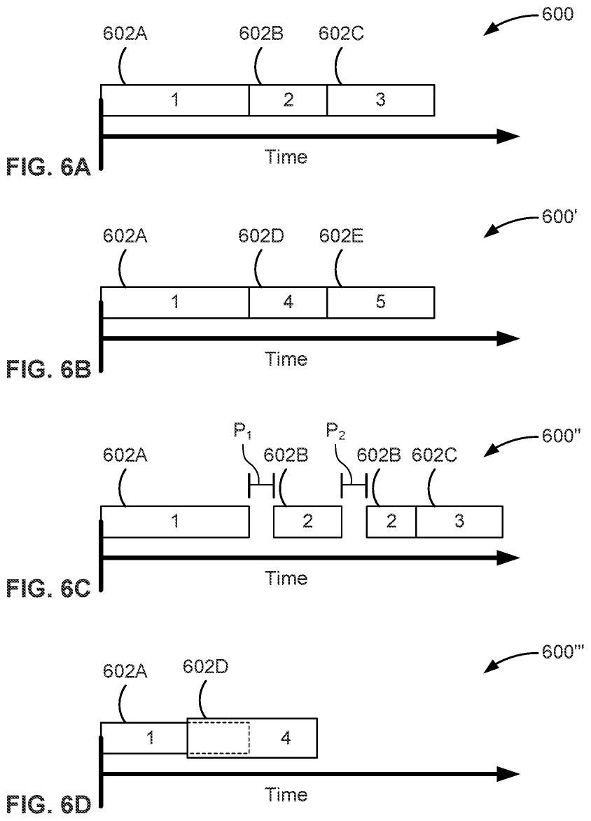

The plurality of activity modules can be stored in the memory device 114 described herein. The plurality of activity modules can include any suitable number of activity modules (e.g., 2, 20, 50, 100, 1,000, 10,000, 100,000, etc.). The activity session includes at least one of the plurality of activity modules, but can include any suitable number of activity modules up to including all of the plurality of activity modules. For example, referring to FIG. 6A, an activity session 600 can include a first activity module 602A, a second activity module 602B, and a third activity module 602C. The activity modules 602A-602C can have the same or different durations and/or difficulties. As one illustrative, non-limiting example, the first activity module 602A can include prompting the user to perform a stretching activity (e.g., to warm up), the second activity module 602B can include prompting the user to perform one or more exercises (e.g., with a difficulty great than the first activity module 602A), and the third activity module 602C can include a cool-down activity.

The one or more activity modules within an activity session can be selected from the plurality of activity modules in a variety of fashions. In some implementations, one or more of the activity modules comprising the activity session are manually selected (e.g., by the user, a medical professional (e.g., therapist, physician), coach, trainer, family, friends, etc.). In other implementations, the plurality of activity modules comprising the activity session are automatically selected. For example, the plurality of activity modules comprising the activity session can be automatically selected based at least in part on information in the user profile described herein (e.g., demographic information such as the age and/or gender of the user, medical information associated with the user, etc.). As another example, the plurality of activity modules comprising the activity session can be automatically selected based at least in part on previously-recorded physiological data and/or previously-recorded motion data associated with the user. As another example, the plurality of activity modules comprising the activity session can be automatically selected based at least in part on current physiological data and/or current motion data associated with the user.

In some implementations, the activity module(s) comprising the activity session are automatically selected from the plurality of modules using a trained machine learning algorithm. The machine learning algorithm can be trained using supervised or unsupervised learning techniques. The training data can be associated with the user or one or more other individuals. In this way, the machine learning algorithm can be said to learn from the training data and adapt to the current user. Once trained, the machine learning algorithm is configured to receive as inputs the user profile associated with the user, previously-recorded physiological data associated with the user, previously-recorded motion data associated with the user, or any combination thereof and select the activity module(s) comprising the activity session as an output. For example, an activity module can be selected for the user based on results for other similarly-situated individuals (e.g., other users in the same age bracket as the user did well with a particular selection and sequence of activity modules).

In some implementations, the activity module(s) comprising the activity session are automatically selected based at least in part on information associated with the user's environment. In such implementations, data from one or more sensors (e.g., radar sensor 132) can be used to analyze the user's environment and select an activity module. Data from the sensor(s) can be used to determine an amount of space the user has to perform activities. For example, if the user only has a few feet between obstacles (e.g., furniture, walls, etc.), an activity module can be selected that does not require movements where the obstacles can interfere. As another example, if an activity module requires use of equipment (e.g., a chair or other furniture, weights, resistance bands, mats/pads, a brace, a sensor, etc.), data from the sensor(s) can be used to identify the presence or absence of such equipment and accordingly select activity module(s). For instance, if an activity module requires a chair and no chair is identified in the environment, that activity module will not be selected as part of the activity session. In these examples, an object recognition algorithm can be used to identify the presence or absence of objects in the user's environment. Further examples of environmental parameters for selecting an activity module can include an ambient temperature, an ambient lighting, an ambient air quality, or any combination thereof. For instance, if the ambient temperature exceeds a predetermined threshold, an activity module with a relatively low intensity can be selected (e.g., to aid in preventing heat-related illness or injury). As another example, if the ambient air quality is below a predetermined threshold (e.g., indicative of poor air quality), an activity module with a relatively low intensity can be selected.

Step 502 of the method 500 includes receiving physiological data associated with the user during the first activity module of the activity session. The physiological data is indicative of one or more physiological parameters of the user such as, for example, heart rate, heart rate variability, cardiac waveform, respiration rate, respiration rate variability, respiration depth, a tidal volume, an inspiration amplitude, an expiration amplitude, an inspiration-expiration ratio, perspiration, temperature (e.g., ambient temperature, body temperature, core body temperature, surface temperature, etc.), blood oxygenation, photoplethysmography, pulse transmit time, blood pressure, or any combination thereof.

Step 503 of the method 500 includes receiving motion data associated with the user during the first activity module of the activity session. The motion data is indicative of movement of the user (or the lack thereof). The motion data can be used, for example, to determine whether the user is performing the activity or activities within the first activity module. For example, the motion data can be compared to previously-recorded motion data (e.g. calibration data) to determine whether the user is performing the activity or activities within the first activity module. In some implementations, the determination whether the user is complying with the first activity module is a binary decision (e.g., yes or no). In other implementations, determining whether the user is complying with the first activity module includes a determination of a degree of compliance (e.g., the user is performing 10%, 50%, 90%, etc. of the first activity module). The motion data can also be used to determine a qualitative performance of the user while performing first activity (e.g., poor, fair, good, excellent, etc.).

In some implementations, the physiological data (step 502) and the motion data (step 503) can be generated or obtained from the same sensor. For example, both the physiological data and the motion data can be generated or obtained from the radar sensor 132 (FIG. 1) described herein. In such implementations, the physiological data and the motion data can be generated or obtained from a sensor (e.g., the radar sensor 132) which is not integrated or coupled to a device that is worn by the user. In other words, the sensor for generating or obtaining the physiological data and/or the motion data can be physically spaced from the user and the clothing of the user, obviating the need for the user to don any device or equipment in order to generate data. In other implementations, the physiological data and the motion data can be generated or obtained from different sensors. In such implementations, the physiological data can be generated by a first sensor while the motion data is generated by a second sensor that is different than the first sensor. For example, the motion data can be generated by the radar sensor 132, while the physiological data can be generated by a pulse oximeter sensor, the PPG sensor 148, and/or the temperature sensor 138 (e.g., one or more of which are integrated in or coupled to a wearable device that is worn by the user during the activity session).

Step 504 of the method 500 includes modifying the activity session subsequent to initiating the activity session (e.g., subsequent to initiating a first activity module). The activity session can be modified in a variety of ways. For example, the activity session can be modified by substituting one or more the activity modules comprising the activity session with one or more alternative activity modules selected from the plurality of activity modules. The activity session can be modified by adding activity modules to the activity session or removing activity modules from the activity session. The activity session can be modified by reordering one or more the activity modules in the activity session. As another example, the activity session can be modified by pausing one or more the activity modules comprising the activity session or adding a pause between a pair of the activity modules comprising the activity session. As further examples, a playback speed of an activity module can be modified (e.g., sped up or slowed down) and/or an audio volume for an activity module can be modified (e.g., increased or decreased). In this manner, the activity session can be adapted to the user based on real-time physiological and/or motion data associated with the user.

For example, referring to FIG. 6B, in one example, the activity session 600 (FIG. 6A) can be modified to form a first modified activity session 600′ by substituting the second activity module 602B with a fourth activity module 602D and/or substituting the third activity module 602C with a fifth activity module 602E. Referring to FIG. 6C, in another example, the activity session 600 (FIG. 6A) can be modified to form a second modified activity session” by adding a first pause P₁ between the first activity module 602A and the second activity module 602B and/or adding a second pause P₂ within the second activity module 602B, thereby splitting the second activity module 602B into a first portion (before the pause) and a second portion (after the pause). As yet another example, referring to FIG. 6D, the activity session 600 (FIG. 6A) can be modified to form a third modified activity session 600′″ by terminating the first activity module 602A early and initiating a fourth activity module 602D.

The activity session can be modified in step 504 based at least in part on at least a portion of the physiological data associated with the user (step 502) during the first activity module, at least a portion of the motion data associated with movement of the user (step 503) during the first activity module, or both. For example, the activity session can be modified based on the physiological data in response a comparison between a physiological parameter associated with the user during the first activity module (e.g., heart rate, heart rate variability, breathing rate, breathing rate variability, body temperature, etc.) and a predetermined threshold. The predetermined threshold can be based on the user profile described herein (e.g., an age of the user, medical conditions, etc.). The physiological data can further be used to predict a physiological parameter associated with the user (e.g., during the next activity module comprising the activity session), for example, based on a trend in the physiological data. For example, if the physiological parameter is a heart rate and the heart rate exceeds a predetermined threshold during the first activity module or is predicted to exceed a predetermined threshold during the first or second activity module, the activity session can be modified to replace the second activity module with a different activity module with a difficulty that is less than the second activity module to aid in preventing the physiological parameter from exceeding the threshold. In another example, the first activity module can be paused responsive to the physiological parameter exceeding the threshold. The pause can have a duration based on continued measuring of the physiological parameter (e.g., the pause continues until the parameter falls below the threshold). Alternatively, the pause can have a predetermined duration (e.g., 10 seconds, 30 seconds, 1 minute, 3 minutes, 10 minutes, etc.). In yet another example, a pause between the first activity module and the next activity module can be added to the activity session responsive to the physiological parameter exceeding the threshold.

As another example, the activity session can be modified based on the motion data in response a comparison between movements of the user and movements required by the first activity module. For example, if the user is not complying with the first activity module (e.g., is not able to complete the requested movements), the activity session can be modified to replace the second activity module with another activity module with a lower difficulty, terminate the first activity module, or remove activity modules from the activity session. Alternatively, the first activity module can be terminated early. Conversely, if the user is complying with the first activity module, the activity session can be modified to replace the second activity module with another activity module with a greater difficultly, stop the first activity module and continue to another activity module, or add additional activity modules to the activity session.

The physiological and motion data can also be used together to modify the activity session. For example, if the motion data indicates that the user is complying with the first activity module and the physiological data indicates that a physiological parameter (e.g., heart rate) is below a threshold, this may indicate that the first activity module is too easy for the user and thus the activity session can be modified (e.g., adding another activity module to the activity session).

In some implementations, the method 500 further includes communicating one or more recommendations to the user during the activity session. The recommendation(s) can be communicated to the user during an activity module, during a pause in an activity module, during a pause between activity modules, or any combination thereof. In some implementations, the recommendation(s) include a recommendation for the user to use additional equipment during the activity session. For example, if the second activity module requires the use of equipment (e.g., weights, resistance bands, braces, etc.), the recommendation can be communicated to the user prior to the second activity (e.g., during a pause between the first activity module and the second activity module) to prompt the user to retrieve and/or prepare the equipment to use. Further, an object recognition algorithm can be used to identify the presence or absence of the equipment, such that the activity session remains paused until the user has the correct equipment. As another example, an activity module is paused due to a physiological parameter (e.g., heart rate, breathing rate, body temperature, etc.) exceeding a threshold, a recommendation can be communicated to the user during the pause to aid in modifying the parameter (e.g., sit down, breathing exercises, drink fluids, etc.).

As described above, in some implementations, the activity session can be modified based at least in part on environmental parameters such as the presence or absence of objects, ambient temperature, ambient lighting, ambient air quality, etc. In such implementations, these environmental parameters can also be used to generate recommendations to the user. For example, if the ambient temperature exceeds a predetermined threshold, a recommendation to lower the room temperature can be communicated to the user (e.g., by adjusting a thermostat). As another example, if the ambient lighting is below a predetermined threshold, a recommendation to turn up the lights can be communicated to the user. As a further example, if it is determined that there is insufficient space for the user to perform an activity module where they are currently standing, a recommendation can be communicated to the user to move to a different location in the room with sufficient space for the activity module.

As described herein, the radar sensor 132 can be used to obtain both the motion data and the physiological data. The physiological data obtained by the radar sensor 132 may be more accurate when the user is standing still or sitting. Thus, in some cases, the radar sensor 132 may not obtain accurate data when the user is moving. To address this issue, a recommendation can be communicated to the user to use an additional sensor responsive to determining that the physiological data is not accurate (e.g., based on a variability, a comparison to expected or historical values, movement of the user, etc.). The additional sensor can be, for example, embedded in or coupled to a wearable device (e.g., smart watch or bracelet). Thus, the user can be prompted to use an additional sensor for obtaining physiological data.

One or more steps of the method 500 can be repeated one or more times. For example, step 502 and step 503 can be repeated for the second activity module subsequent to the first activity module. Accordingly, the activity session can be modified as described herein at any time during the activity session. The data received in step 502 and step 503 can be stored over multiple iterations (e.g., in the memory) so to improve the effectiveness of future modifications to the activity session. For example, repeating these steps can be used to train a machine learning algorithm used to determining when and how to modify the activity session during step 504. In this way, the method 500 described herein can adaptively learn which activity module(s) are optimal based on the current user and create activity sessions accordingly.

While the system 100, method 400, and method 500 have been described herein with reference to a single user, more generally, the system 100, the method 400, and the method 500 can be used with a plurality of users simultaneously (e.g., 2 users, 5 users, 10 users, 20 users, etc.). For example, the system 100, method 400 and the method 500 can be used in a group therapy setting. Further, while the systems and methods have been described herein as relating to delivering therapy to a user, more generally, the systems and methods described herein can be used in non-therapy applications, such as, for example, exercise applications. In such implementations, the systems and methods described herein can be used to determine whether the user is performing the exercise (e.g., to monitor compliance) and/or determine whether a physiological parameter of the user exceeds a predetermined threshold during the exercise (e.g., for safety reasons).

One or more elements or aspects or steps, or any portion(s) thereof, from one or more of any of claims 1-83 below can be combined with one or more elements or aspects or steps, or any portion(s) thereof, from one or more of any of the other claims 1-83 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
causing first media content to be displayed on a display device, the first media content including a first prompt to perform a first activity;
receiving, from one or more sensors, data associated with the user, the data including (i) motion data associated with movement of the user and (ii) physiological data associated with the user;
determining whether the user is performing the first activity based at least in part on the motion data associated with the user, the physiological data associated with the user, or both;
determining a first physiological parameter associated with the user subsequent to the first prompt based at least in part on the physiological data associated with the user;
determining whether the first physiological parameter exceeds a predetermined threshold; and
generating an alert responsive to (i) determining that the first physiological parameter exceeds the predetermined threshold, (ii) determining that the user is not performing the first activity, or (iii) both (i) and (ii).

2. (canceled)

3. The method of claim 1, further comprising responsive to determining that the user is not performing the first activity, causing a second prompt to perform the first activity to be displayed on the display device.

4. The method of claim 1, further comprising causing an operation of the display device to be modified responsive to (i) determining that the first physiological parameter exceeds the predetermined threshold, (ii) determining that the user is not performing the first activity, or (iii) both (i) and (ii).

5. The method of claim 4, wherein the modification includes pausing the media content that is displayed on the display device.

6. The method of claim 4, further comprising:
determining a second physiological parameter associated with the user based at least in part on the physiological data subsequent to the modification;
determining whether the second physiological parameter exceeds the predetermined threshold; and
causing the media content to resume on the display device responsive to determining that the physiological parameter is below the predetermined threshold.

7. The method of claim 4, wherein the modification includes causing a second prompt to perform a second activity to be displayed on the display device, wherein the second activity is selected based at least in part on the determination whether the user was performing the first activity, the determined first physiological parameter, or both.

8. (canceled)

9. The method of claim 1, further comprising causing an indication of the determined first physiological parameter to be displayed on the display device, wherein the indication of the determined first physiological parameter is overlaid on or adjacent to the displayed media content.

10. (canceled)

11. The method of claim 1, wherein the first physiological parameter includes a heart rate, a heart rate variability, a respiration rate, a respiration rate variability, or any combination thereof.

12. (canceled)

13. The method of claim 12, further comprising determining that the user completed the first activity based at least in part on the data, responsive to determining that the user completed the first activity, causing second media content to be displayed on the display device, the second media content including a second prompt to perform a second activity, the second activity being different than the first activity.

14. (canceled)

15. The method of claim 1, further comprising determining an identity of the user based at least in part on the data from at least one of the one or more sensors.

16. The method of claim 1, wherein the first media content includes one or more video images, one or more still images, audio, or any combination thereof.

17. The method of claim 1, wherein the first activity is a therapy session.

18-34. (canceled)

35. A system comprising:
a one or more sensors configured to generate data associated with a user;
a memory storing machine-readable instructions and media content; and
a control system including one or more processors configured to execute the machine-readable instructions to:
cause a display device to display a first portion of the media content, the first portion of the media content including a first prompt for performing a first activity;
determine whether the user is performing the first activity based at least in part on the data generated by the one or more sensors;
determine a first physiological parameter associated with the user based at least in part on the data generated by the one or more sensors;
determine whether the first physiological parameter exceeds a predetermined threshold; and
cause an operation of the display device to be modified responsive to determining that the first physiological parameter exceeds the predetermined threshold.

36. The system of claim 35, wherein the one or more sensors include a radar sensor.

37. (canceled)

38. The system of claim 36, further comprising a housing, wherein the radar sensor, the control system, and the memory are coupled to or integrated in the housing.

39. (canceled)

40. The system of claim 38, further comprising an interface configured to be received within a port of the display device to communicatively couple the display device and the memory.

41-43. (canceled)

44. The system of claim 36, further comprising a secondary device including a second one of the one or more sensors that is different than the radar sensor.

45-52. (canceled)

53. A method comprising:
prompting a user to initiate an activity session comprising a first activity module and a second activity module, the first activity module and the second activity module being selected from a plurality of activity modules, the second activity module being subsequent to the first activity module;
receiving physiological data associated with the user during the first activity module;
receiving motion data associated with movement of the user during the first activity module;
modifying the activity session based at least in part on at least a portion of the physiological data associated with the user, at least a portion of the motion data associated with movement of the user, or both, wherein the modifying the activity session includes (i) pausing the first activity module for a first duration, (ii) pausing the activity session between the first module and the second module for a second duration, (iii) substituting the second activity module with a third activity module selected from the plurality of modules, or (iv) any combination thereof.

54. The method of claim 53, wherein the first activity module has a first difficulty and the second activity module has a second difficulty that is different than the first difficulty.

55. (canceled)

56. The method of claim 53, wherein the first activity module and the second activity module are selected from the plurality of modules based at least in part on a user profile associated with the user, previously-recorded physiological data associated with the user, previously-recorded motion data associated with the user, or any combination thereof.

57. (canceled)

58. The method of claim 56, wherein the first activity module and the second activity module are selected from the plurality of modules using a trained machine learning algorithm, the trained machine learning algorithm being configured to (i) receive as inputs the user profile associated with the user, previously-recorded physiological data associated with the user, previously-recorded motion data associated with the user, or any combination thereof and (ii) output the first activity module and the second activity module.

59. (canceled)

60. The method of claim 53, wherein the modifying the activity session includes removing the second activity module from the activity session and terminating the activity session subsequent to the first activity module.

61. The method of claim 53, wherein the first duration, the second duration, or both are determined based at least in part on at least a portion of the physiological data associated with the user, at least a portion of the motion data associated with movement of the user, or both.

62-65. (canceled)

66. The method of claim 63, further comprising determining a first physiological parameter associated with the user during the first activity module based at least in part on the physiological data, and determining whether the first physiological parameter exceeds a predetermined threshold.

67. The method of claim 66, wherein the modifying the activity session includes (i) pausing the first activity module or (ii) terminating the first activity module responsive to determining that the first physiological parameter exceeds the predetermined threshold.

68. (canceled)

69. The method of claim 67, further comprising causing a recommendation to be communicated to the user, wherein the modifying the activity session includes pausing the first activity module and the recommendation is communicated to the user while the first activity module is paused.

70. The method of claim 67, further comprising causing a recommendation to be communicated to the user, wherein the modifying the activity session includes pausing the activity session between the first activity module and the second activity module and the recommendation is communicated to the user between the first activity module and the second activity module.

71. The method of claim 67, further comprising causing a recommendation to be communicated to the user, wherein the recommendation includes a recommendation for the user to use additional equipment during the first activity module, the second activity module, or both.

72. (canceled)

73. The method of claim 53, wherein the modifying activity session includes pausing the first activity module, selecting another activity module from the plurality of activity modules, prompting the user to initiate the another activity module, and resuming the first activity module subsequent to the another activity module.

74-83. (canceled)
